(19) United States
(12) Patent Application Publication
(10) Pub. No.: US 2006/0009661 A1
(43) Pub. Date: Jan. 12, 2006

(54) Continuous method for the production of sugar alcohols

(75) Inventors: Jan-Dirk Arndt, Mannheim (DE); Katrin Klass, Mannheim (DE); Frederik van Laar, Limburgerhof (DE); Stephan Herwig, Ludwigshafen (DE); Jochem Henkelmann, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/538,265

(22) PCT Filed: Dec. 3, 2003
(86) PCT No.: PCT/EP03/13632

§ 371 Date: Jun. 10, 2005

(30) Foreign Application Priority Data
Dec. 11, 2002 (DE) 102 58 089.8
Nov. 6, 2003 (DE) 103 52 336.7

(51) Int. Cl. C07C 29/14

(52) U.S. Cl. 568/863

(57) ABSTRACT

A continuous process for preparing sugar alcohols by catalytic hydrogenation of an aqueous solution of a saccharide, which forms the corresponding sugar alcohol on hydrogenation, in the presence of a ruthenium catalyst which is obtainable by: i) single or multiple treatment of an amorphous silicon-dioxide-based support material with a halogen-free aqueous solution of a low-molecular-weight ruthenium compound and subsequent drying of the treated support material at below 200° C., ii) reducing the solid obtained in i) with hydrogen at from 100 to 350° C., step ii) being carried out immediately after step i), which comprises, before the hydrogenation, bringing the aqueous saccharide solution to be hydrogenated into contact with the support material.

The present invention relates to a continuous process for preparing sugar alcohols by catalytic hydrogenation of suitable saccharides.

The sugar alcohol sorbitol is prepared industrially by catalytic hydrogenation of glucose, fructose, sucrose or invert sugar (see H. Schiweck et al. “Sugar Alcohols” in Ullmann's Encyclopedia of Industrial Chemistry, 5th ed. on CD-ROM). For this purpose, the catalysts used to date have primarily been nickel catalysts, for example supported nickel catalysts or Raney nickel. There have also been several reports on the use of ruthenium-containing catalysts for this purpose. Generally, the ruthenium catalysts are supported catalysts that comprise ruthenium on an oxidic support or an organic support such as carbon.

U.S. Pat. No. 4,380,680, U.S. Pat. No. 4,487,980, U.S. Pat. No. 4,413,152 and U.S. Pat. No. 4,471,144 describe the preparation of sorbitol by catalytic hydrogenation of glucose, in which catalysts are used that comprise ruthenium on a support material which is stable under hydrothermal conditions. Hydrothermal support materials which are proposed are alpha-alumina (U.S. Pat. No. 4,380,680), titanium(IV) oxide (U.S. Pat. No. 4,487,980), titanium(IV)-halide-treated alumina (U.S. Pat. No. 4,413,152) and theta-alumina (U.S. Pat. No. 4,471,144).

U.S. Pat. No. 4,503,274 discloses catalysts for hydrogenating glucose to sorbitol which are prepared by impregnating a hydrothermally stable support with an aqueous ruthenium halide solution and subsequently hydrogenating the solid at from 100 to 300° C.

U.S. Pat. No. 3,963,788 describes hydrogenating corn starch hydrolyzates to sorbitol in the presence of ruthenium catalysts in which the ruthenium was supported by an aluminosilicate-based specific zeolite. U.S. Pat. No. 3,963,789 proposes as support for ruthenium catalysts crystalline aluminosilicate clays, in particular montmorillonite.

FR-A 2526782 describes the use of a ruthenium chloride prepared by reacting sodium chloride and ruthenium via Na₂RuCl₆ for preparing silica-supported ruthenium catalysts for hydrogenating mono- and oligosaccharides, for example for preparing sorbitol.

The processes known from the prior art for preparing sorbitol by hydrogenation in the presence of ruthenium catalysts, owing to the only moderate activity of the catalysts, give sorbitol only with moderate space-time yields, based on the catalyst used. In view of the high costs of ruthenium, therefore, the economic efficiency of these processes leaves something to be desired. In addition, the selectivities of the catalysts are not sufficient, so that additional expenditure is required in isolating the products of value. In particular, epimerization of the hydroxyl groups is frequently observed.

It is an object of the present invention, therefore, to provide a continuous process for preparing sugar alcohols by catalytic hydrogenation of the corresponding saccharides which form the desired sugar alcohols on hydrogenation, which process avoids the abovementioned disadvantages and, in particular, gives the desired sugar alcohols with improved space-time yields and in which fewer by-products are produced and which permits longer catalyst service lives.

This object has surprisingly been achieved by a continuous process for preparing sugar alcohols by catalytic hydrogenation of an aqueous solution of a saccharide, which forms the corresponding sugar alcohol on hydrogenation, in the presence of a ruthenium catalyst which is obtainable by:
i) single or multiple treatment of an amorphous silicon-dioxide-based support material with a halogen-free aqueous solution of a low-molecular-weight ruthenium compound and subsequent drying of the treated support material at below 200° C.,
ii) reducing the solid obtained in i) with hydrogen at from 100 to 350° C.,
   step ii) being carried out immediately after step i), which comprises, before the hydrogenation, bringing the aqueous saccharide solution to be hydrogenated into contact with the support material.

Suitable saccharides comprise in principle all known tetroses, pentoses, hexoses and heptoses, more precisely not only aldoses but also ketoses and also their disaccharides and oligosaccharides. The monosaccharides which can be used in the inventive process comprise, for example: erythrose, threose, ribose, arabinose, xylose, lyxose, allose, altrose, mannose, gulose, idose, galactose, talose, erythrulose, ribulose, xylulose, psicose, tagatose, glucose, fructose and gulose, more precisely not only the D form but also the L form. Invert sugar which is obtainable by hydrolysis out of sucrose is also suitable. Examples of disaccharides are: maltose, isomaltose, lactose, cellobiose, melobiose and sucrose.

Suitable mono- and oligosaccharides for the inventive hydrogenation process which may be mentioned are, in particular, the monosaccharides mannose for the preparation of mannitol, galactose for the preparation of dulcitol (galactitol) and xylose for the preparation of xylitol, preferably the D form of the monosaccharides, and also the disaccharides maltose for the preparation of maltitol, isomaltulose (palatinose) for the preparation of isomaltitol and lactose for the preparation of lactitol.

A preferred starting material for the preparation of the sugar alcohol sorbitol is glucose and also glucose-rich syrup such as corn starch hydrolyzates, wheat starch hydrolyzates and potato starch hydrolyzates. The preparation of D-sorbitol by hydrogenation of the D form of the abovementioned monosaccharides is of particular interest.

However, the other mono- and oligosaccharides mentioned can also be hydrogenated in the presence of the inventive ruthenium catalysts to give the corresponding sugar alcohols. The hydrogenation of aldoses leads to sugar alcohols which have with respect to the OH groups the same configuration as the sugar used, and the hydrogenation of furanoses generally leads to mixtures of two diastereomeric sugar alcohols which differ only in the configuration of the carbon atom which carries the carbonyl function in the furanose. The respective pure sugar alcohol is generally isolated from this mixture without problems.

The mono- and oligosaccharides can be used as such or as mixtures, the starting materials preferably being used in pure form.

It is presumed that the high activity of the catalysts used in the inventive process can be attributed to the particularly good distribution of the ruthenium on the surface of the support material and to the substantial absence of halogen in the support material.

Owing to the preparation, the ruthenium is present in the inventive catalysts as metallic ruthenium.

Electron microscope studies (TEM) of the catalysts have found that the ruthenium is present on the support material in atomic dispersion form and/or in the form of ruthenium particles which are present virtually exclusively, that is to say more than 90%, preferably more than 95%, based on the number of visible particles, as isolated particles having diameters less than 10 nm, in particular less than 7 nm. In other words, the catalyst comprises essentially no, that is to say less than 10%, in particular less than 5%, ruthenium particles and/or agglomerates of ruthenium particles having diameters greater than 10 nm. Owing to the use of halogen-free ruthenium precursors and solvents in the preparation, the chlorine content of the inventively used catalysts is in addition less than 0.05% by weight (<500 ppm), based on the total weight of the catalyst.

An essential constituent of the catalysts used in the inventive process is the amorphous silicon dioxide-based support material. The term amorphous in this context means that the content of crystalline silicon dioxide phases makes up less than 10% of the support material. The support materials used for preparing the catalysts can, however, have superstructures which are formed by regular disposition of pores in the support material.

Support materials which come into consideration are in principle all amorphous silicon dioxide types that consist of at least 90% by weight silicon dioxide, the remaining 10% by weight, preferably no more than 5% by weight, of the support material also being able to be another oxidic material, for example MgO, CaO, TiO₂, ZrO₂, Fe₂O₃ or alkali metal oxide. Obviously, the support material used is also halogen-free, that is to say the halogen content is less than 500 ppm. Preferably the support material comprise no more than 1% by weight, and in particular no more than 0.5% by weight, and in particular no detectable amounts (<500 ppm), of aluminum oxide, calculated as Al₂O₃. In a preferred embodiment support materials are used which comprise less than 500 ppm of Fe₂O₃. The proportion of alkali metal oxide generally results from the preparation of the support material, and can be up to 2% by weight. Frequently, it is less than 1% by weight. Suitable supports are also alkali metal oxide-free supports (<0.1% by weight). The proportion of MgO, CaO, TiO₂ or of ZrO₂ can constitute up to 10% by weight of the support material and is preferably no more than 5% by weight. However, support materials comprising no detectable amounts of these metal oxides (<0.1% by weight) are also suitable.

Preference is given to support materials which have a specific surface area in the range from 50 to 700 m²/g, in particular in the range from 80 to 600 m²/g, and especially in the range from 100 to 600 m²/g (BET surface area as defined in DIN 66131). Among the pulverulent support materials, in particular preference is given to those whose specific (BET) surface area is in the range from 200 to 600 m²/g. In the case of support material in the form of shaped bodies, the specific surface area is in particular in the range from 100 to 300 m²/g.

Suitable amorphous silicon-dioxide-based support materials are familiar to those skilled in the art and are commercially available (see, for example, O. W. Flörke, “Silica” in Ullmann's Encyclopedia of Industrial Chemistry 5th ed. on CD-ROM). They can either be of natural origin or synthetically prepared. Examples of suitable amorphous silicon-dioxide-based support materials are kieselguhr, silica gels, pyrogenic silicic acid and precipitated silicic acid. In a preferred embodiment of the invention, the catalysts comprise silica gels as support materials.

Depending on the embodiment of the inventive process, the support material can have differing forms. If the process is configured as a suspension process, to prepare the inventive catalysts, usually, the support material is used in the form of a finely divided powder. The particle size of the powder particles is preferably in the range from 1 to 200 μm, and in particular in the range from 10 to 100 mm. When the catalyst is used in fixed-bed catalysts, usually shaped bodies of the support material are used, which are obtainable, for example, by extrusion or tableting and which can have, for example, the shape of spheres, tablets, cylinders, strands, rings or hollow cylinders, stars and the like. The dimensions of these shaped bodies are usually in the range from 1 mm to 25 mm. Frequently, catalyst strands are used having strand diameters of from 2 to 5 mm and strand lengths of from 2 to 25 mm.

The ruthenium content in the catalysts can be varied over a wide range. Generally, it is at least 0.1% by weight, preferably at least 0.2% by weight, and frequently does not exceed a value of 10% by weight, in each case based on the weight of the support material. Preferably, the ruthenium content is in the range from 0.2 to 7% by weight, and in particular in the range from 0.4 to 0.5% by weight.

The ruthenium catalysts used in the inventive process are generally prepared by firstly treating the support material with a halogen-free aqueous solution of a low-molecular-weight ruthenium compound, termed (ruthenium) precursor hereinafter, in such a manner that the desired amount of ruthenium is taken up by the support material. This step is also termed impregnating hereinafter. The support thus treated is then dried at the above-specified temperatures. If appropriate, the resultant solid is then again treated with the aqueous solution of the ruthenium precursor and again dried. This procedure is repeated until the amount of ruthenium compound taken up by the support material corresponds to the desired ruthenium content in the catalyst.

The support material can be treated or impregnated in various ways depending in a known manner on the shape of the support material. For example, the support material can be sprayed or rinsed with the precursor solution, or the support material can be suspended in the precursor solution. For example, the support material can be suspended in the aqueous solution of the ruthenium precursor and after a certain time can be filtered off from the aqueous supernatant. The ruthenium content of the catalyst can then be controlled in a simple manner via the amount of liquid taken up and the ruthenium concentration of the solution. The support material can also be impregnated, for example, by treating the support with a defined amount of the aqueous solution of ruthenium precursor corresponding to the maximum amount of liquid which the support material can take up. For this purpose, the support material can be sprayed, for example, with the amount of liquid. Suitable apparatuses for this are the apparatuses customarily used for mixing liquids with solids (see Vauck/Müller, Grundoperationen chemischer Verfahrenstechnik [Unit operations of chemical engineering], 10th edition, Deutscher Verlag für Grundstoffindustrie, 1994, pp. 405 et seq.), for example tumbler dryers, impregnating drums, drum mixers, blade mixers and the like. Monolithic supports are usually rinsed with the aqueous solutions of the ruthenium precursor.

The aqueous solutions used for impregnating are according to the invention halogen-free, that is to say they contain no halogen or less than 100 ppm halogen. The ruthenium precursors used are therefore only those ruthenium compounds which do not comprise chemically bound halogen and which are sufficiently soluble in the aqueous solvent. These include, for example, ruthenium(III) nitrosylnitrate (Ru(NO)(NO₃)₃), ruthenium(III) acetate and the alkali metal ruthenates(IV) such as sodium ruthenate(IV) and potassium ruthenate(IV).

Aqueous here means water and mixtures of water containing up to 50% by volume, preferably no more than 30% by volume, and in particular no more than 10% by volume, of one or more water-miscible organic solvents, for example mixtures of water with C₁-C₄-alkanols such as methanol, ethanol, n-propanol or isopropanol. Frequently, water is used as sole solvent. The aqueous solvent will frequently additionally comprise at least one halogen-free acid, for example nitric acid, sulfuric acid, phosphoric acid, or acetic acid, preferably a halogen-free mineral acid, in the solution for stabilizing the ruthenium precursor. In many cases, therefore, a halogen-free mineral acid diluted with water, for example nitric acid diluted to half-concentrated, is used as solvent for the ruthenium precursor. The concentration of ruthenium precursor in the aqueous solutions of course depends on the amount of ruthenium precursor to be added and the absorption capacity of the support material for the aqueous solution and is generally in the range from 0.1 to 20% by weight.

Drying can be performed by the customary processes of solids drying, complying with the abovementioned temperatures. Complying with the inventive upper limit of drying temperatures is important for the quality, that is to say the activity, of the catalyst. Exceeding the above-specified drying temperatures leads to a significant loss of activity. Calcining the support at relatively high temperatures, for example above 300° C., or even 400° C., as proposed in the prior art, is not only superfluous, but also has a disadvantageous effect on the catalyst activity.

The ruthenium-precursor-impregnated solid is usually dried at atmospheric pressure, reduced pressure also being able to be used to promote drying. Frequently, to promote drying, a gas stream, for example air or nitrogen, is passed over or through the material to be dried.

The drying time of course depends on the desired degree of drying and the drying temperature and is generally in the range from 2 h to 30 h, preferably in the range from 4 h to 15 h.

Preferably, the treated support material is dried until the content of water or of volatile solvent constituents, before reduction ii), makes up less than 5% by weight, in particular no more than 2% by weight, and particularly preferably no more than 1% by weight, based on the total weight of the solid. The specified percentages by weight relate to the loss of weight of the solid determined at 300° C., a pressure of 1 bar and for a time of 10 min. In this manner, the activity of the inventive catalysts can be further increased.

Preferably, the drying is performed with agitation of the precursor-solution-treated solid, for example by drying the solid in a rotary kiln or a rotating ball furnace. In this manner the activity of the inventive catalysts can be further increased.

The solid obtained after drying is converted into its catalytically active form according to the invention by hydrogenating the solid at the above-specified temperatures in a manner known per se.

For this purpose, the support material, at the above-specified temperatures, is brought into contact with hydrogen or a mixture of hydrogen and an inert gas. The hydrogen partial pressure is of minor importance for the result of the reduction and can be varied in the range from 0.2 bar to 1.5 bar. Frequently the catalyst material is hydrogenated at atmospheric pressure of hydrogen in the hydrogen stream. Preferably, the hydrogenation takes place with agitation of the solid obtained in i), for example by hydrogenating the solid in a rotary kiln or a rotating ball furnace. In this manner the activity of the inventive catalysts can be further increased.

After the hydrogenation, the catalyst, to improve the handleability, can be passivated in a known manner, for example by briefly treating the catalyst with an oxygen-containing gas, for example air, but preferably with an inert gas mixture containing from 1 to 10% by volume of oxygen.

In the inventive process, the saccharide is preferably hydrogenated by hydrogenating an aqueous solution of the respective saccharide, or in the case of invert sugar as starting material, of the saccharide mixture. “Aqueous” here is defined as above. Expediently, water is used as sole solvent, which may comprise small amounts of a preferably halogen-free acid for setting the pH. In particular, the monosaccharide is used as aqueous solution having a pH in the range from 4 to 10, especially in the range from 5 to 7.

The saccharide concentration in the liquid phase can in principle be chosen freely, and is frequently in the range from 10 to 80% by weight, and preferably in the range from 15 to 50% by weight, based on the total weight of the solution.

The saccharide solution, before the hydrogenation, that is to say before it comes into contact with the ruthenium catalyst, is brought into contact with the support material. This is so that the saccharide solution becomes saturated in support material, that is to say especially silicon dioxide, and as a result dissolves less support material out of the catalyst, which has an advantageous effect on the lifetime (service life) of the catalyst. The saccharide solution can be contacted with the support material in a plurality of ways, for example by suspending the pulverulent support material in the saccharide solution, or by transferring the saccharide solution through shaped bodies made of support material.

Transfer of the saccharide solution through silica rods is a particularly preferred embodiment of the inventive method, in particular if the solution is forced under pressure through tubes packed with extruded silica rods.

A further advantage of the inventive method is yielded by any oligomeric sugars still present in the saccharide solution being retained when forcing the saccharide solution through the silica rods, and thus the purity of sugar alcohol formed being increased. This is to be observed, in particular, when starch hydrolyzates are used as saccharide.

The actual hydrogenation is usually performed in a similar manner to the known hydrogenation processes for preparing sugar alcohols, as described in the prior art mentioned at the outset. For this purpose the liquid phase comprising the saccharide is brought into contact with the catalyst in the presence of hydrogen. The catalyst here can either be suspended in the liquid phase (suspension procedure) or the liquid phase is passed through a fluid catalyst bed (fluid-bed procedure) or a fixed catalyst bed (fixed-bed procedure). The hydrogenation can be either continuous or batchwise. Preferably, the inventive process is carried out in trickling reactors by the fixed-bed procedure. The hydrogen can be passed over the catalyst either cocurrently with the solution of the starting material to be hydrogenated, or countercurrently.

Suitable apparatuses for carrying out hydrogenation by the suspension procedure and also for hydrogenation on a fixed catalyst bed are known from the prior art, for example from Ullmanns Enzyklopädie der Technischen Chemie [Ullmann's Encyclopedia of Industrial Chemistry], 4th Edition, Volume 13, pp. 135 et seq., and from P. N. Rylander, “Hydrogenation and Dehydrogenation” in Ullmann's Encyclopedia of Industrial Chemistry, 5th ed. on CD-ROM.

Generally, the hydrogenation is carried out at elevated hydrogen pressure, for example at a hydrogen partial pressure of at least 10 bar, preferably at least 20 bar, and in particular at least 40 bar. Generally, the hydrogen partial pressure does not exceed 500 bar, in particular 350 bar. Particularly preferably, the hydrogen partial pressure is in the range from 40 to 200 bar. The reaction temperatures are generally at least 40° C., and frequently do not exceed 250° C. In particular, the hydrogenation process is carried out at from 80 to 150° C.

Owing to the high catalyst activity, comparatively small amounts of catalyst are required based on the starting material used. Thus, in the batchwise suspension procedure, generally less than 1 mol %, for example from 10⁻³ mol % to 0.5 mol % of ruthenium, based on 1 mol sugar is used. In the continuous form of the hydrogenation process, usually the starting material to be hydrogenated is passed over the catalyst at a rate of from 0.05 to 2 kg/(l(catalyst)*h), in particular at a rate of from 0.07 to 0.7 kg/(l(catalyst)*h).

In the inventive process, a solution of the sugar alcohol in the aqueous solvent respectively used is produced, from which the sorbitol can be obtained by known processes (see H. Schiweck et al. “Sugar Alcohols” in Ullmann's Encyclopedia of Industrial Chemistry, 5th ed. on CD-ROM). In the case of the aqueous reaction mixtures preferably obtained, the sugar alcohol can be isolated, for example, by evaporation with subsequent crystallization (DE-A 2350690, EP-A 32288, EP-A 330352) or spray-drying (DK 33603, DD 277176). If necessary, the catalyst is removed in advance by customary processes and the reaction solution is subjected to decolorization using suitable filter aids and/or treatment with ion exchangers to remove metal ions, gluconates or other organic acids.

When invert sugar or fructose is used, in addition to sorbitol, obviously also mannitol is formed. If pure sugar alcohols are desired, sorbitol can be isolated from the resultant reaction mixtures, for example, by selective crystallization.

The inventive process is distinguished by the high space-time yields achieved and, when glucose is used as starter material, also by high product selectivity. In addition, the inventive process is distinguished by particularly high service lives of the ruthenium catalysts, as a result of which the process becomes particularly economically attractive.

Obviously, the catalysts used in this process, when the activity decreases, can be regenerated according to methods known to those skilled in the art which are customary for noble metal catalysts such as ruthenium catalysts. Those which may be mentioned here are, for example, treating the catalyst with oxygen, as described in BE 882279, treatment with dilute halogen-free mineral acids, as described in U.S. Pat. No. 4,072,628, or treatment with hydrogen peroxide, for example in the form of aqueous solutions having a content of from 0.1 to 35% by weight, or treatment with other oxidizing substances, preferably in the form of halogen-free solutions. Usually, the catalyst is rinsed with a solvent, for example water, after the reactivation and before renewed use.

The examples below serve for more detailed explanation of the invention:

I Preparation of the Catalysts

1. Protocol A: Pulverulent, Halogen-Free Catalyst, Non-Calcined.

A defined amount of the respective support material was impregnated with the maximum amount of a solution of ruthenium(III) nitrosylnitrate in water, which could be absorbed by the respective support material. The maximum amount absorbed by the respective support material had been determined in advance on the basis of an authentic sample. The concentration of the solution was in each case such as to result in the desired concentration of ruthenium in the support material.

The resultant solid was then dried for 13 h at 120° C. in a rotary ball oven. The residual water content was less than 1% by weight.

The resultant solid was reduced at atmospheric pressure in a hydrogen stream at 300° C. in a rotary ball furnace for 4 h. After cooling and rendering inert with nitrogen, the catalyst was passivated by passing over 5% by volume of oxygen in nitrogen for a period of 120 min.

2. Protocol B: Pulverulent, Halogen-Free Catalyst, Calcined.

The catalyst was prepared in a similar manner to protocol A, but the solid obtained after drying was heated at 400° C. in an air stream for 4 h before the hydrogenation.

3. Protocol C: Pulverulent, Halogen-Containing Catalyst, Non-Calcined.

The catalyst was prepared in a similar manner to protocol A, but instead of ruthenium(III) nitrosylnitrate, ruthenium(III) chloride was used.

4. Protocol D: Rod-Shaped, Halogen-Free Catalyst, Non-Calcined.

A defined amount of cylindrical support-material strands (diameter 4 mm, length from 3 to 10 mm) was impregnated in water with the maximum amount of a solution of ruthenium(III) nitrosylnitrate which could be absorbed by the respective support material. The maximum amount absorbed by the respective support material had been determined in advance on the basis of an authentic sample. The concentration of the solution was in each case such as to result in the desired concentration of ruthenium in the support material.

The resultant impregnated rods were then dried for 13 h at 120° C. in a rotary ball furnace. The residual water content was less than 1% by weight.

The resultant dried rods were reduced in a rotary ball furnace for 4 h at 300° C. in a hydrogen stream at atmospheric pressure. After cooling and rendering inert with nitrogen, the resultant catalyst was passivated by passing over 5% by volume of oxygen in nitrogen for a period of 120 min.

5. Protocol E: Rod-Shaped, Halogen-Containing Catalyst, Non-Calcined.

The catalyst was prepared in a similar manner to protocol D, but ruthenium(III) chloride was used instead of ruthenium(III) nitrosylnitrate.

II Continuous Hydrogenation of Corn Starch Hydrolyzate in the Presence of a Fixed-Bed Catalyst for Producing Sorbitol

A reaction unit consisting of a main reactor with circulation and also a post-reactor is charged with the ruthenium catalyst prepared under I.

An aqueous solution of corn starch hydrolyzate having a glucose concentration of 40% is passed under pressure through a tube packed with extruded silica rods. This solution is then passed into the main reactor which had an overhead temperature of from 80 to 130° C., and thereafter passed through the post-reactor, the overhead temperature of which had been adjusted to the bottom temperature of the main reactor. The hydrogenation was performed at a pressure of 140 bar.

The process yields a conversion rate of 99.8% and a selectivity based on sorbitol of 99.3%.

III Continuous Hydrogenation of Xylose in the Presence of a Fixed-Bed Catalyst for Producing Xylitol

A reaction unit consisting of a main reactor with circulation and also a post-reactor is charged with the ruthenium catalyst prepared under I.

An aqueous solution of xylose (source: Aldrich, purity 99.6%) having a concentration of 30% is passed under pressure through a tube packed with extruded silica rods. This solution is then passed into the main reactor which had an overhead temperature of from 80 to 130° C., and thereafter passed through the post-reactor, the overhead temperature of which had been adjusted to the bottom temperature of the main reactor. The hydrogenation was performed at a pressure of 90 bar.

The process yields a conversion rate of 99.8% and a selectivity based on xylitol of 98.5%.

1. A continuous process for preparing sugar alcohols by comprising catalytically hydrogenating an aqueous solution of a saccharide, forming the corresponding sugar alcohol by hydrogenation of the saccharide, in the presence of a ruthenium catalyst which is obtained by:
i) single or multiple treatment of an amorphous silicon-dioxide-based support material with a halogen-free aqueous solution of a low-molecular-weight ruthenium compound and subsequent drying of the treated support material at below 200° C.,
ii) reducing the solid obtained in i) with hydrogen at from 100 to 350° C.,
step ii) being carried out immediately after step i), which comprises, before the hydrogenation, bringing the aqueous saccharide solution to be hydrogenated into contact with the support material.

2. The process according to claim 1, wherein the sugar alcohol prepared is sorbitol or xylitol.

3. The process according to claim 1, wherein the aqueous saccharide solution is a wheat starch hydrolyzate or corn starch hydrolyzate.

4. The process according to claim 1, wherein the aqueous saccharide solution, before the hydrogenation, is forced through silica rods.
